(19) United States
(12) Patent Application Publication
(10) Pub. No.: US 2015/0238068 A1
(43) Pub. Date: Aug. 27, 2015

(54) ENDOSCOPE WITH PIVOTABLE LENS SYSTEM

(71) Applicant: OLYMPUS WINTER & IBE GMBH, Hamburg (DE)

(72) Inventors: Jens ROSE, Hamburg (DE); Martin WIETERS, Hamburg (DE)

(73) Assignee: OLYMPUS WINTER & IBE GMBH, Hamburg (DE)

(21) Appl. No.: 14/710,142

(22) Filed: May 12, 2015

Related U.S. Application Data
(63) Continuation of application No. PCT/EP2013/003077, filed on Oct. 14, 2013.

(30) Foreign Application Priority Data
Nov. 12, 2012 (DE) 102012220578.0

(51) Int. Cl. A61B 1/00

(52) U.S. Cl. CPC A61B 1/00183; A61B 1/00096

(57) ABSTRACT

An endoscope including an endoscope shaft closed by a cover glass; an optical system arranged in the endoscope shaft, the optical system including at least one prism located proximally to the cover glass; a prism holder for holding the optical system and mounted so as to be pivotable about a rotation axis perpendicular to a longitudinal axis of the endoscope shaft, wherein the prism holder has at least a portion having a circular circumference with a toothed arrangement; an elongated adjustment element movable along the longitudinal axis of the endoscope shaft and having a mating toothed arrangement for engagement with the toothed arrangement of the prism holder, the mating toothed arrangement being formed at least on a distal portion; wherein at least the distal portion of the adjustment element is bendable such that it is guided in a curved guide channel formed at least partially around the prism holder.

### CROSS-REFERENCE TO RELATED APPLICATION

The present application is a continuation of PCT/EP2013/003077 filed on Oct. 14, 2013, which is based upon and claims the benefit to DE 10 2012 220 578.0 filed on Nov. 12, 2012, the entire contents of each of which are incorporated herein by reference.

### BACKGROUND

1. Field

The present application relates to an endoscope with a pivotable lens system, comprising an endoscope shaft closed at the distal end by a cover glass, an optical system arranged in the endoscope shaft, and at least one prism, which is located between the cover glass and the optical system and is arranged in a prism holder that is mounted so as to be pivotable about a rotation axis perpendicular to the longitudinal extent of the endoscope shaft, wherein the prism holder has at least in part a circular circumference with a toothed arrangement into which an elongated adjustment element, reversibly movable in the direction of the axial longitudinal extent of the endoscope shaft, engages via a complementary toothed arrangement formed at least in part on said element. The present application further relates to a use.

2. Prior Art

Endoscopes, and in particular video endoscopes, in which the light of an operative field entering at a distal tip of an endoscope shaft of the endoscope is directed through an optical system onto one or more images sensors, are known in different designs. Thus, there are endoscopes with a direct view, of a so-called 0° viewing direction, or endoscopes with a lateral viewing direction, which have for example a lateral viewing direction of 30°, 45°, 70° or the like differing from the 0° viewing direction. The named degree numbers hereby mean the angle between the central viewing axis and the longitudinal axis of the endoscope shaft. There are also endoscopes or respectively video endoscopes with an adjustable lateral viewing direction, in which the viewing angle, i.e. the deviation from the direct view, is adjustable. The generic endoscopes for the present application comprise so-called “pivoting prisms” or “pivoting prism arrangements” for adjusting the viewing angle.

Besides an adjustment of the viewing angle, i.e. of the deviation from the direct view, the viewing direction, i.e. the azimuth angle, can also be adjusted around the longitudinal axis of the endoscope shaft, in that the endoscope is rotated in its entirety around the longitudinal axis of the endoscope shaft.

A slip-free change in the viewing angle is realized in different manners. For this, some endoscopes use prisms that are arranged behind a curved cover glass and are arranged on a prism holder rotatable around a rotational axis. These direct the light from different viewing angles to an optical system in the endoscope. An axially aligned, longitudinally extended adjustment element, which is designed for example as a toothed rack and engages in a toothed arrangement on the prism holder, serves to pivot the prism. A direct, hinged connection with a hinge on the prism holder is also known. Finally, a rotation of the corresponding adjustment element can also be used if the adjustment element has a screw thread, which engages in teeth on the prism holder. Corresponding examples are known from US 2010/0030031 A1.

In these solutions, the slip-free rotation of the adjustment element with a screw thread is complex to create, while the control with an axially linearly movable control element either requires a lot of space on the distal tip in order to achieve a large viewing angle, or the adjustable adjustment angle is very limited when there is little space available.

### SUMMARY

In contrast, an object is to enable a compact endoscope with a simple, slip-free viewing angle adjustment in a large viewing angle area.

This object is solved by an endoscope with a pivotable lens system, comprising an endoscope shaft closed at the distal end by a cover glass, an optical system arranged in the endoscope shaft, and at least one prism, which is located between the cover glass and the optical system and is arranged in a prism holder that is mounted so as to be pivotable about a rotation axis perpendicular to the longitudinal extent of the endoscope shaft, wherein the prism holder has at least in part a circular circumference with a toothed arrangement into which an elongated adjustment element, reversibly movable in the direction of the axial longitudinal extent of the endoscope shaft, engages via a complementary toothed arrangement formed at least in part on said element, which is further developed in that the adjustment element is bendable and is guided in a guide channel formed at least in part around the prism holder.

The endoscope combines the advantage of the toothed rack solution, namely a large, achievable pivoting angle or respectively viewing angle area, with a compact structure, since a rigid toothed rack no longer needs to be housed. The bendable adjustment element is inserted distally into the already present space behind the cover glass. A slip-free transfer of a translatory movement into a rotational movement in a small space results.

The adjustment element can be designed with shear and tensile strength. In this manner, a linear translation movement of the adjustment element is translated in a play-free manner into a pivoting of the prism holder with the prism.

The adjustment element can be designed at least in part as a fixable rod, chain or belt. The proximal section, which bends around the prism holder, is thereby designed in a flexible manner transverse to its longitudinal extent.

The complementary toothed arrangement can have toothed receivers, which are designed as a toothed arrangement, as a row of closed toothed receiver openings or as a row of laterally open toothed receiver openings. The rows of closed or laterally open toothed receiver openings allow a thin and flexible and thus compact designed of the adjustment element, while the design as a toothed arrangement also permits a particularly high shear and tensile strength in addition to flexibility. The version with a toothed arrangement can be designed as an evolvent toothing, which allows a particularly even and smooth transfer of power and movement. The recesses or respectively toothed receiver openings do not have to be closed laterally. For easier production, they can also be produced as a comb. A row of openings designed as notches or recesses can also be provided for example on both sides around a center bar. In this case, the complementary toothed arrangement has two lateral rows of teeth. During the movement of the adjustment element with the complementary toothed arrangement, the rotationally mounted prism holder is rotated around its axis through the engagement of the teeth.

The teeth of the toothed arrangement can be beveled on the circumference of the prism holder in the direction of engagement. This is already the case for an evolvent toothing and is also useful in the case of another toothing, in particular in a variant with an adjustment element that has a row of closed or laterally open toothed receiver openings. It is hereby achieved that the engagement of the subsequent tooth is enabled although the least possible to no play occurs due to the tooth with the toothed receiver openings. The forced guidance in the guide channel (at least in sections) also contributes to this.

The adjustment element can also be made of a sheet metal and/or an elastomer or comprises a sheet metal and/or an elastomer. The elastomer as such or respectively as an admixture or part of the adjustment element has, as does the sheet metal, the necessary flexibility, also the beneficial shear and tensile strength. Its design with toothed arrangement or toothed receiver openings also flows into the selection of the material. The toothed rack can be designed flexibly in the direction of engagement, i.e. transverse to the longitudinal extent. In the case of a design as sheet metal or with a sheet metal, it is achieved that a thin sheet metal with recesses is used. Due to the small thickness of the sheet metal, it is easily and very elastically deformable in the desired direction. Moreover, the elastic area can be expanded further if necessary through a special material selection, for example with Nitinol. The recesses can be introduced into the sheet metal easily and exactly by means of laser cutters, for example.

However, due to the easy deformability in the direction of engagement, it is necessary that the sheet metal or respectively the adjustment element is guided in the direction of engagement since it could otherwise be easily pushed out of engagement with the toothed arrangement of the prism holder. Moreover, the guide ensures the most constant possible lever arm to the rotation axis of the rotated prism holder and thus for a constant transmission of the gear.

In a further development, the adjustment element is forcibly guided at least in sections in the endoscope shaft and in the distal area behind the cover glass, in particular without lateral play. The guide channel serves this purpose at least in sections. It is thus possible to also effectuate a movement in tight spaces without wasting space unnecessarily. The forced guidance includes the fact that the adjustment element bends or respectively kinks behind the prism holder. It can thus only concern a short section, at which the power transmission takes place, or a longer section around the prism holder. The material selection is such that the cover glass is not scratched, or respectively the forced guidance can also be such that the adjustment element does not touch the cover glass.

It is useful if a non-guided flexible area of the adjustment element, on which the actuation force acts, which is arranged proximal to the guide, is held as low as possible since the flexible area can bulge due to the flexibility during actuation in the distal direction. No rotational movement or a delayed rotational movement, comparable with slippage, can hereby occur. It is also useful if, in the non-deflected state, the length of the flexible part behind the guide has the length of the necessary stroke for the completely required rotation. A more rigid area can follow after this. A small addition can be added to this distance for balancing the tolerances and from other tolerance-generating structures and influences such as edge rounding. This addition can be as small as possible. The adjustment element thus has a flexible distal part, the length of which corresponds with the length of the required stroke plus a tolerance compensation. The adjustment element is then designed less flexibly proximal to the flexible distal part. The prism holder can be pivotable by more than 60°, in particular more than 80°. A large viewing angle area is thus adjustable.

The connection between the adjustment element and prism holder can be, except for unavoidable tolerances in the processing and guiding of the toothed arrangement and the complementary toothed arrangement, designed in a slip-free manner. This highlights the connection according to the present application for example with respect to a friction connection, which has a certain slip.

The object can also be solved through a use of a bendable adjustment element with a complementary toothed arrangement designed distally at least in part in a previously described endoscope for pivoting a prism arranged distally or respectively on the distal end of an endoscope shaft behind a cover glass. This use allows the distal area of the endoscope shaft in the endoscope to be designed in a compact manner. It is thus possible to arrange the cover glass very close to the prism and vice versa so that the prism has the same configuration of cover glass in front of it in each pivot angle or respectively viewing angle. This is not possible for some telescopes and some lens systems when a rigid toothed rack is used, which protrudes far beyond the prism holder during the adjustment of large lateral viewing angles and accordingly requires a lot of space distally.

Further characteristics will become apparent from the description of embodiments together with the claims and the included drawings. The disclosed embodiments can fulfill individual characteristics or a combination of several characteristics.

### BRIEF DESCRIPTION OF THE DRAWINGS

The embodiments are described below, without restricting the general idea of the invention, using exemplary embodiments with reference to the drawings, whereby we expressly refer to the drawings with regard to all details according to the invention that are not explained in greater detail in the text. The figures shown in:

FIG. 1 illustrates a schematic cross-sectional representation through the distal part of a conventional endoscope known in the art,

FIG. 2 illustrates a schematic cross-sectional representation through the distal part of an endoscope according to a first embodiment,

FIGS. 3a) and 3b) illustrate schematic perspective representations of an endoscope tip in a second embodiment,

FIGS. 4a) and 4b) illustrate schematic perspective representations of an endoscope tip in a third embodiment and

FIGS. 5a)-5d) illustrate schematic perspective representations of an endoscope tip in a fourth embodiment.

### DETAILED DESCRIPTION

In the drawings, the same or similar types of elements and/or parts are provided with the same reference numbers so that a re-introduction is omitted.

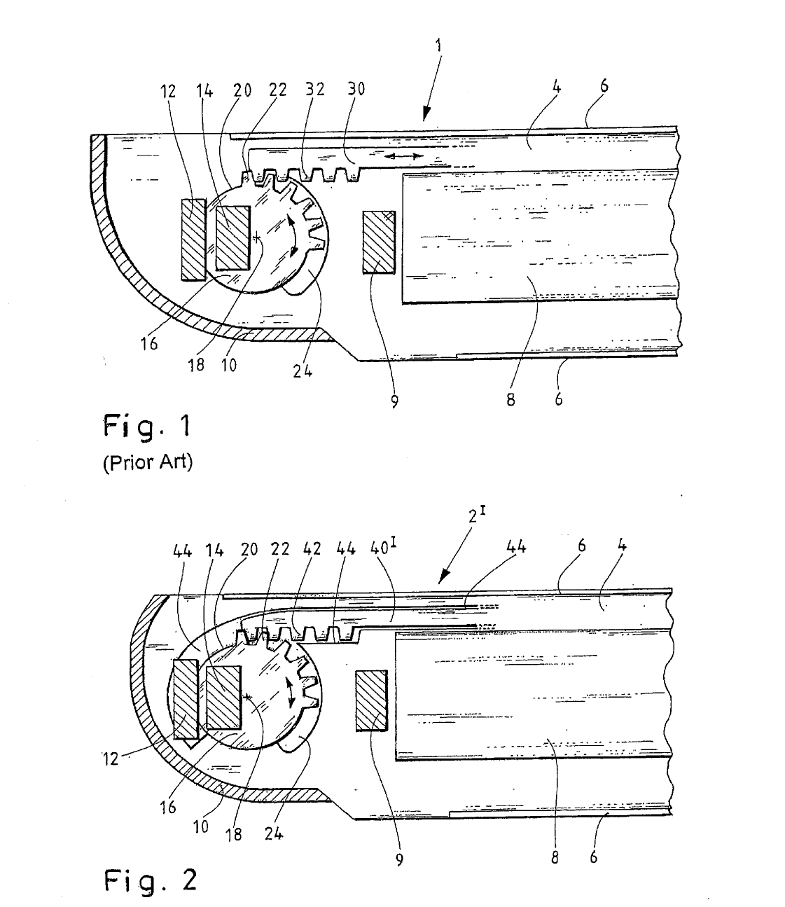

FIG. 1 shows in detail and schematically in cross-section a conventional endoscope 1 known in the art with a pivot prism arrangement. The distal end of the endoscope shaft 4, which is closed to the outside by a jacket tube 6, is formed by a cover glass 10, which hermetically seals the interior. A prism holder 16, which is pivotable around a rotation axis 18, which progresses transverse, in particular perpendicular, to the longitudinal extent of the endoscope shaft 4, is arranged behind the cover glass 10. The prism holder 16 holds a prism 14 as well as an also pivoted entry lens 12. An annular hollow space 24 is also shown, which is present so that the prism holder 16 with its toothed arrangement 22 has space to turn. Further details of the housing on the distal tip of the endoscope shaft 4 of the endoscope 1 are not shown for clarity's sake.

After light has passed through the cover glass 10, the entry lens 12 and the prism, it arrives at an exit lens 9, which transfers the light to the optical system 8 of the endoscope 1. This can be a lens combination with a video sensor or respectively an image sensor or a set of inverter lenses, which transfer the light up to the proximal end and if applicable to a camera head and/or an ocular.

The geometry of prism 14, prism holder 16 and cover glass 10 is broken. In the case of a direct view, as shown in FIG. 1, the distance between cover glass 10 and entry lens 12 is greater than in the case of a view perpendicularly downwards. The angles of the boundary surfaces of the cover glass 10 with respect to the entry lens 12 are also different in the different positions. This effect is caused by the mechanism, with which the prism holder 16 is pivotable. The prism holder 16 is designed mainly circularly with at least one circular circumferential section 20, which has in section a circumferential toothed arrangement 22. A toothed arrangement 32 of a rigid toothed rack 30 engages in this toothed arrangement 22, which imparts a linear axial movement, shown by a straight double arrow, proximally, i.e. from the handle. Through the axial linear movement of the toothed rack 30, the prism holder 16 is rotated, which is shown by a curved double arrow on the prism holder 16.

FIG. 1 shows the direct view, wherein the toothed rack 30 is shown in its position that is pulled back the furthest. In order to pivot the viewing angle up to 90°, the toothed rack 30 must be moved further linearly and axially in the direction of the cover glass 10. In the end state, the toothed rack 30 thereby protrudes considerably over the prism holder 16. For this reason, the cover glass 10 is provided with a slight bend in the extension of the toothed rack 30 in order to create sufficient space for the toothed rack 30.

FIG. 2 shows schematically a cross-section of an endoscope 2^{I} according to a first embodiment. It differs from the known endoscope 1 according to FIG. 1 among other things in the design of the adjustment element 40^{I}, which is a bendable adjustment element 40^{I} with a distal toothed arrangement 42. It is forcibly guided in a guide channel 44, which is designed proximally to the prism holder 16 and is then arranged distally and concentrically to the rotation axis 18 around the prism holder 16. The flexible or respectively the bendable adjustment element 40^{I} is thus guided around the prism holder 16 during the linear movement in the guide channel 44 and requires less space distally in the axial direction. For this reason, the cover glass 10 of the endoscope 2 is henceforth designed bent towards the top, ideally also concentrically with the rotation axis 18 of the prism holder 16.

Through this solution, the light at any viewing angle passes through a similar configuration of boundary surfaces so that a uniform rendering of the environment is ensured at any viewing angle. Moreover, in this manner, the same available pivoting area can also be used efficiently, which is not the case in FIG. 1, where the cover glass 10 is not arranged symmetrically around the rotation axis 18.

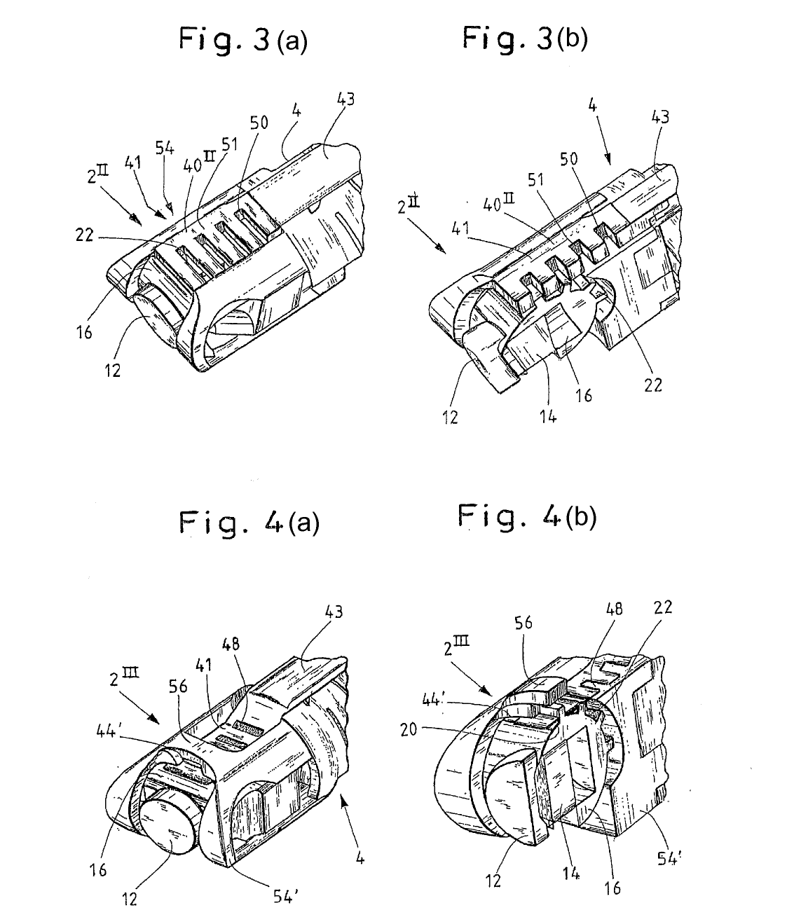

FIGS. 3a) and 3b) show schematically in detail a tip of an endoscope 2^{II} in a perspective representation angled from above, wherein outer jacket tubes are omitted for a better overview. A prism holder 16, which holds an entry lens 12 and a prism 14 shown in cross-section in FIG. 3b), is arranged in a distal housing 54 on the distal top of the endoscope 2^{II}. The prism holder 16 has a toothed arrangement 22 on its circumference. The viewing direction is a 0° viewing direction, i.e. straight ahead.

The bendable adjustment element 40^{II} has a flexible distal section 41 with a complementary toothed arrangement 42, which is designed as a row of laterally open toothed receiver openings 50. The teeth of the toothed arrangement 22 of the prism holder 16 engage in the toothed receiver openings 50. A more rigid section 43 of the adjustment element 40^{II} is connected proximally.

FIG. 3b) shows again in detail the engagement of the teeth of the toothed arrangement 22 of the prism holder 16 in the laterally open toothed receiver openings 50 of the comb structure 51. The more rigid proximal section 43 can be made of a more rigid material than the bendable distal section 41 or, as shown in FIGS. 3a and 3b), covered completely with a more rigid material layer. The flexible lower layer in the sections 41 and 43 hereby merges into each other. The thickness of the adjustment element 40^{II} is selected to be comparatively thick so that a forced guidance can be achieved for example in that the shown inner structure with the housing 54 is inserted into an enveloping tube.

FIGS. 4a) and 4b) show in a schematic perspective representation a further exemplary embodiment of a distal tip of an endoscope 2″ in a direct view or respectively in a 0° viewing direction. In this case, the flexible distal section 41 of the adjustment element 40^{III} is thin and has a row of closed toothed receiver openings 48, which engage in succession with the teeth of the toothed arrangement 22 of the prism holder 16. In this exemplary embodiment in relation to the flexible section 41, the more rigid proximal section 43 has a reinforcing position of a more rigid material, but which does not cover the more flexible material in the full width.

The distal housing 54′ has a bridge 56, under which the thin distal section 41 of the adjustment element 40^{III} is forcibly guided. A secure engagement between the toothed receiver openings 48 and the teeth of the toothed arrangement 22 is thus secured in the area, in which the tangent on the circular circumferential section 20 of the prism holder 16 is parallel to the longitudinal extent of the endoscope shaft. The adjustment element 40^{III} is free again distally to the bridge 56.

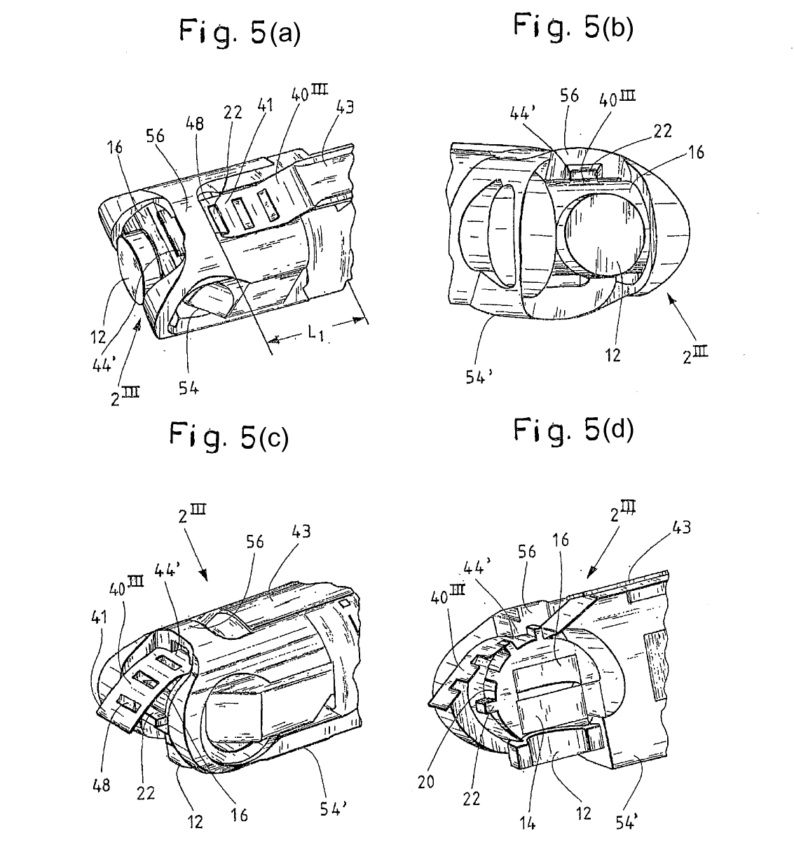

FIGS. 5a) to 5d) show the endoscope 2^{III} according to FIG. 4 in further detail. FIG. 5a) shows the direct view from an upper perspective. The length of the free part of the flexible section 41 labeled with L₁ from the beginning of the bridge 56 to the transition to the more rigid section 43 is shown. This takes into account the maximum necessary deflection of the prism holder 16 up to the maximum adjustable viewing angle.

FIG. 5b) shows a front view distally, which clearly shows that the thin adjustment element 40^{III} is forcibly guided between the inside of the bridge 56 and the surface of the prism holder 16.

FIG. 5c) shows the distal tip of the endoscope 2^{III} in a 90° viewing direction. Therefore, the entry lens 12 is pointed downwards. In order to adjust the viewing angle, the adjustment element 40^{III} has been axially displaced by a total of four tooth engagements or respectively toothed receiver openings 48. The adjustment element 40^{III} is no longer forcibly guided distally to the bridge 56 and sticks up slightly for reasons of rigidity. In this case, the material of the adjustment element 40^{III} is selected such that contact with a cover glass 10 does not scratch or otherwise damage it. The same is shown again in cross-section in FIG. 5d).

The specific complementary arrangement of openings and teeth is freely selectable. The flexible section can have, for example, a center bar with rows of lateral notches on both sides as an opening, or the openings can be distributed in any manner, even irregularly, as long as the teeth are arranged in a manner that is suitably complementary to this structure.

All named characteristics, including those taken from the drawings alone and also individual characteristics, which are disclosed in combination with other characteristics, are considered alone and in combination as essential for the invention. Embodiments according to the invention can be realized by individual characteristics, or a combination of several characteristics.

### LIST OF REFERENCE NUMBERS

1 Endoscope

2^{II}-2^{III} Endoscope

4 Endoscope shaft

6 Jacket tube

8 Optical system

9 Exit lens

10 Cover glass

12 Entry lens

14 Prism

16 Prism holder

18 Rotation axis

20 Circular circumferential section

22 Toothed arrangement

24 Hollow space

30 Toothed rack

32 Toothed arrangement

40^{I}-40^{III} Bendable adjustment element

41 Flexible section

42 Complementary toothed arrangement

43 More rigid section

44 Guide channel

46 Toothed arrangement

48 Closed toothed receiver opening

50 Laterally open toothed receiver opening

51 Comb structure

54, 54′ Distal housing

56 Bridge

What is claimed is:

1. An endoscope with a pivotable lens system, the endoscope comprising:
an endoscope shaft closed at a distal end by a cover glass;
an optical system arranged in the endoscope shaft, the optical system including at least one prism located proximally to the cover glass;
a prism holder for holding the optical system and mounted so as to be pivotable about a rotation axis perpendicular to a longitudinal axis of the endoscope shaft, wherein the prism holder has at least a portion having a circular circumference with a toothed arrangement;
an elongated adjustment element movable along the longitudinal axis of the endoscope shaft, the elongated adjustment element having a mating toothed arrangement for engagement with the toothed arrangement of the prism holder, the mating toothed arrangement being formed at least on a distal portion of the elongated adjustment element;
wherein at least the distal portion of the elongated adjustment element is bendable such that at least the distal portion is guided in a curved guide channel formed at least partially around the prism holder.

2. The endoscope according to claim 1, wherein the adjustment element is designed with shear and tensile strength.

3. The endoscope according to claim 1, wherein the adjustment element is configured at least partially as a flexible rod, chain or belt.

4. The endoscope according to claim 1, wherein the mating toothed arrangement has toothed receivers arranged as one of a row of closed toothed receiver openings or as a row of laterally open toothed receiver openings.

5. The endoscope according to claim 1, wherein the toothed arrangement includes beveled teeth on the circular circumference of the prism holder in a direction of engagement with the mating toothed arrangement.

6. The endoscope according to claim 1, wherein at least the distal portion of the elongated adjustment element is formed of one or both of a sheet metal and an elastomer.

7. The endoscope according to claim 1, wherein the distal portion of the elongated adjustment element is forcibly guided in the curved guide channel proximally of the cover glass.

8. The endoscope according to claim 7, wherein a length of the distal portion of the elongated adjustment element corresponds with a length of a required stroke elongated adjustment element along the longitudinal axis plus a tolerance compensation.

9. The endoscope according to claim 8, wherein portions of the elongated adjustment element proximal to the distal portion has a flexibly which is less flexible than the distal end.

10. The endoscope according to claim 1, wherein the prism holder is pivotable by more than 60° about the rotation axis.

11. The endoscope according to claim 1, wherein the prism holder is pivotable by 80° about the rotation axis.

12. A method of changing a viewing direction for an endoscope, the method comprising:
rotatably disposing a prism holder for holding an optical system so as to be pivotable about a rotation axis perpendicular to a longitudinal axis of an endoscope shaft, wherein the prism holder has at least a portion having a circular circumference with a toothed arrangement;
movably disposing an elongated adjustment element in the endoscope shaft so as to be movable along the longitudinal axis of the endoscope shaft, the elongated adjustment element having a mating toothed arrangement for engagement with the toothed arrangement of the prism holder, the mating toothed arrangement being formed at least on a distal portion of the elongated adjustment element; and
bending at least the distal portion of the adjustment element upon a distal movement of the elongated adjustment element such that at least the distal portion is guided in a curved guide channel formed at least partially around the prism holder.
